(19) United States
(12) Patent Application Publication
(10) Pub. No.: US 2021/0316288 A1
(43) Pub. Date: Oct. 14, 2021

(54) HYBRID MATERIAL AND METHOD FOR THE PRODUCTION THEREOF

(71) Applicants: Renal BACKOV, Bordeaux (FR); Clement SANCHEZ, Bures Sur Yvette (FR); Herve DELEUZE, Le Barp (FR); Simona UNGUREANU, Jud Iasi (RO)

(72) Inventors: Renal BACKOV, Bordeaux (FR); Clement SANCHEZ, Bures Sur Yvette (FR); Herve DELEUZE, Le Barp (FR); Simona UNGUREANU, Jud Iasi (RO)

(21) Appl. No.: 12/527,161

(22) PCT Filed: Feb. 14, 2008
(86) PCT No.: PCT/FR08/00193

§ 371 Date: Feb. 7, 2013

(30) Foreign Application Priority Data
Feb. 14, 2007 (FR) 0701077
Feb. 14, 2008 (FR) PCT/FR2008/000193

(51) Int. Cl. B01J 31/12; B01J 23/44; B01J 35/04; B01J 35/10; B01J 35/00; B01J 37/02; C07C 2/86; B01D 53/81; C02F 1/28; B01D 53/72

(52) U.S. Cl. CPC B01J 31/125; C02F 2101/322; B01J 23/44; B01J 35/04; B01J 35/1076; B01J 35/1061; B01J 35/109; B01J 35/0013; B01J 37/0219; B01J 37/0217; B01J 37/0228; B01J 37/0244; B01J 37/0236; C07C 2/86; B01D 53/81; C02F 1/285; B01D 53/72; B01J 2231/005; B01J 2231/4261; B01J 2231/4211; B01J 2531/005; C07C 2523/44; C07C 2531/06; B01D 2253/204; B01D 2257/7027; B01J 31/126

(57) ABSTRACT

The invention relates to a material in the form of a cellular solid monolith consisting of an inorganic oxide polymer. Said monolith comprises macropores which have an average size d_{A} of 4 μm to 50 μm, mesopores that have an average size d_{E} of 20 to 30 Å, and micropores which have an average size d₁ of 5 à 10 Å, said pores being interconnected. The inorganic oxide polymer has organic groups R of formula —(CH₂)ₙ—R¹, wherein 0≤n≤5, and R¹ is selected from among a thiol group, a pyrrole group, an amino group having one or more optional, optionally substituted alkyl, alkylamino, or aryl substituents, an alkyl group, or a phenyl group optionally having an alkyl-type substituent R². The disclosed material can be used as a substrate for a metal catalyst and for decontaminating liquid or gaseous media.

The present invention relates to a hybrid material, a method for the preparation thereof and its use as a catalyst support and/or for the decontamination of liquid or gaseous media.

The hybrid material according to the invention forms part of materials having a high specific surface area and an organized structure, namely a cellular structure having several types of porosity.

A “hybrid” material is understood to mean a material carrying inorganic functional groups and organic functional groups.

Such materials may find applications in many fields such as heterogeneous catalysis, solid phase extraction, filtration, electronics, optics or acoustics.

A material with an organized structure is known from FR-2 852 947, which is in the form of a monolith made of an inorganic material. A monolith is understood to mean a solid subject having an average size of at least 1 mm. The inorganic material consists of a polymer of an inorganic oxide, for example a polymer obtained from tetraethoxysilane Si(OEt)₄. This material is obtained by a high internal phase inverse emulsion polymerization method and possesses three degrees of porosity: microporosity, mesoporosity and macroporosity. The presence of surface silanol groups, which have a certain degree of acidity, makes it possible to use this material in heterogeneous catalysis, but only in acid catalysis.

Materials are also known from the publication by A: Desforges et al., Adv. Func. Mater., 2005, 15, 1689-1695, having a porous organic Matrix based on styrene and divinylbenzene, functionalized by organic groups. These materials are obtained in the form of a monolith by a high internal phase inverse emulsion polymerization method. The monolith has both a macroporous character and a mesoporous character. Such materials are used satisfactorily as catalyst supports, for example a palladium catalyst in the form of nanoparticles. However, catalytic reactions carried out with such materials can be performed at a maximum temperature of 80° C., a temperature above which the organic matrix deteriorates and loses its monolithic character. Such a disadvantage thus limits the use of these materials to a considerable extent.

A continual need therefore exists for finding novel materials having improved properties prepared with known materials, and that are capable of acting as catalyst supports, notably in the rapidly expanding field of heterogeneous catalysis based on supported noble metals.

The inventors have been able to develop a material in the form of a monolith consisting of an inorganic polymer that is functionalized by special organic groups. They have discovered that such a material surprisingly exhibits high performance as a catalyst support, in the field of heterogeneous catalysis. Indeed, notably when it is associated under certain conditions with palladium nanoparticles, this material reveals itself to be more effective than known catalysts based on palladium on active carbon, and this at temperatures that may extend up to approximately 200° C. Moreover, this material is also efficient in other applications, notably the decontamination of liquid or gaseous media.

A material according to the present invention is a solid cellular monolith consisting of a polymer of an inorganic oxide, characterized in that:
a) said cellular monolith has macropores having a mean size d_{A} from 4 μm to 50 μm, mesopores having a mean size d_{E} from 20 to 30 Å and micropores having a mean size d_{I} from 5 to 10 Å, said pores being interconnected;
b) the inorganic oxide polymer carries organic R groups corresponding to the formula —(CH₂)ₙ—R¹ in which 0≤n≤5, and R¹ represents:
   a thiol group,
   a pyrrolyl group C₄H₃N—, linked by nitrogen to the —(CH₂)ₙ— group,
   an amino group amino that may carry one or more possibly substituted alkyl, alkylamino or aryl substituents,
   an alkyl group (preferably having 1 to 5 carbon atoms) or
   a phenyl group that may carry an alkyl substituent, notably a methyl group.

A monolith is understood to mean an object of which the smallest of its dimensions is greater than one millimeter.

The inorganic oxide is an oxide of one or more elements, at least one of these elements being of the type capable of forming an alkoxide. As examples of elements capable of forming an alkoxide, mention may be made of Si, and metals such as Ti, Zr, Th, Nb, Ta, V, W and Al.

The inorganic oxide may be a simple oxide, and it then consists of an oxide of one of the above elements. The inorganic oxide may also be a mixed oxide of at least two elements, and at least one of the elements is chosen from the above elements, it being possible for the other elements to be notably B or Sn.

An inorganic polymer consisting of a polymer of silicon oxide or of a mixed oxide of silicon is particularly preferred.

In one embodiment, the inorganic oxide polymer carries a single type of R group. In another embodiment, the inorganic oxide polymer carries at least two different types of R group.

In particular, the organic group R may be:
a 3-mercaptopropyl group;
a 3-aminopropyl group;
a 3-pyrrolylpropyl group;
a N-(2-aminoethyl)-3-aminopropyl group;
a 3-(2,4 dinitrophenylamino)propyl group;
a phenyl or benzyl group; or
a methyl group.

A material according to the invention may be obtained by a method in which an emulsion is prepared by adding an oily phase to an aqueous solution of surfactant, at least one tetra-alkoxide (noted herein after by TAM) precursor of the inorganic oxide polymer is added to the aqueous surfactant solution, before or after preparing the emulsion, the reaction mixture is allowed to stand until the precursor condenses, and then the mixture is dried so as to obtain a monolith, said method being characterized in that at least one precursor alkoxide carrying an organic R group is added (noted hereinafter by the compound AMR).

In one embodiment, AMR is introduced into the aqueous surfactant solution before the oily phase is added.

In another embodiment, AMR is introduced into the oil phase that is then added to the aqueous TAM solution to form the emulsion.

In a 3^{rd} embodiment, the inorganic monolith obtained from the aqueous surfactant solution and TAM after drying is impregnated with a solution of AMR.

In the 1^{st} and 2^{nd} embodiments, the hybrid monolith obtained at the end of the drying step may advantageously be subjected to a heat treatment, preferably carried out at a temperature of between 140° C. and 180° C. (for example for a period of 6 hours with a temperature rise of 2° C. per minute) with the aim of consolidating the monolith.

The mass ratio AMR/TAM is preferably less than 20/80. If the proportion of AMR is greater than 20%, the mechanical strength of the monolith is weakened.

Implementation of the first embodiment makes it possible to obtain a hybrid material in which the R groups are distributed statistically on the surface as well as in the core of the material.

Implementation of the second embodiment makes it possible to obtain a hybrid material having R groups distributed substantially on the surface of the material.

Implementation of the third embodiment makes it possible to obtain a hybrid material on which the R groups are present exclusively on the surface.

TAM is a tetra-alkoxide of a tetravalent element, possibly in a hydrolyzed and/or partially condensed form. Silicon tetra-alkoxides are particularly preferred, in particular tetramethoxysilane and tetraethoxysilane (TEOS). A silicate or any other substituted oligomer may also be used.

The compound AMR is advantageously chosen from trialkoxysilanes bearing an R group as defined above.

As an example, mention may be made of
(3-mercapto-propyl) trimethoxysilane,
(3-aminopropyl)triethoxysilane,
N-(3-trimethoxysilylpropyl)pyrrole,
3-(2,4 dinitrophenylamino)propyltriethoxysilane,
N-(2-aminoethyl)-3-aminopropyltrimethoxysilane,
phenyltriethoxysilane and
methyltriethoxysilane.

The oily phase may consist of dodecane, or a silicone oil.

The surfactant compound may be a cationic surfactant chosen notably from tetradecyltrimethylammonium bromide (TTAB), dodecyltrimethylammonium bromide or cetyl-trimethylammonium bromide. When the surfactant compound is cationic, the reaction medium is brought to a pH below 3, preferably below 1. Cetyl-trimethylammonium bromide is particularly preferred.

The surfactant composition may also be an anionic surfactant chosen from sodium dodecylsulfate, sodium dodecylsulfonate and sodium dioctylsulfosuccinate (AOT). When the surfactant composition is anionic, the reaction medium is brought to a pH above 10.

The surfactant compound may finally be a non-ionic surfactant chosen from surfactants with an ethoxylated head, and nonylphenols. When the surfactant composition is non-ionic, the reaction medium is brought to a pH above 10 or below 3, preferably below 1.

For the preparation of a hybrid material carrying more than one type of R group, the various precursors of the various R groups may be introduced simultaneously into the reaction medium, or introduced during two successive steps.

When the precursors of the various R groups are introduced into the reaction medium during two successive steps, the first step may consist of the addition of an AMR′ compound according to the previously mentioned first or second variants, and the second step may consist of the subsequent grafting of an AMR″ compound (according to the previously mentioned third variant). In this case, it is understood that R′ and R″ each corresponds to the definition of R given above, R′ being different from R″.

A material according to the invention is particularly useful as a support for a metal catalyst, such as Pd, Au or Pt.

A supported catalyst is prepared by a method consisting of impregnating a monolith according to the invention with a solution of a catalyst metal precursor, and then of reducing the precursor.

The catalyst metal precursor is preferably an acetate or a chloride, for example Pd(CH₃COO)₂, PdCl₂, PtCl₄ or AuCl₄.

The precursor is used in the form of a solution in a solvent, for example THF, THF/water, acetone/water or ethanol/water, according to the hydrophilic/lipophilic balance of the polymer forming the foam.

When the supported catalyst is intended to be used for a reaction in an oxidizing medium, a supported catalyst is preferably used prepared in the presence of a phosphine, for example triphenylphosphine.

A supported catalyst according to the present invention is of use notably for a Suzuki-Myaura reaction. The Suzuki-Myaura reaction is a carbon-carbon coupling reaction that makes it possible to form a biphenyl compound from an aryl iodide and an aryl hydrobromide.

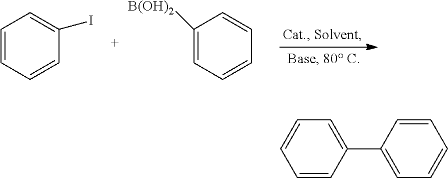 [SMILES: Ic1ccccc1 ; OB(O)c1ccccc1 ; c1ccc(-c2ccccc2)cc1]

A supported catalyst according to the present invention is also of use for the reaction of a Z—Ar—BH(NiPr₂) compound with an Ar—Z′ compound in the presence of a Pd(O) catalyst, a base and water so as to obtain an Ar—Ar compound, according to the following equation of the reaction:

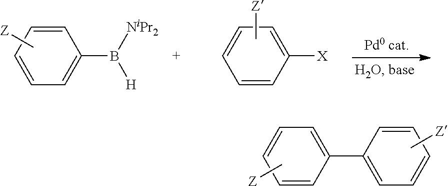

According to the reaction medium in which the catalyst has to be used, its hydrophilic or hydrophobic character may be adjusted by the choice of the Z or Z′ groups. For example, a Z or Z′ group of the alkyl or phenyl type increases the hydrophobic character of the hybrid material. A material carrying SH groups and/or NH₂ groups is particularly useful as a metal catalyst support, since the presence of a non-binding doublet on sulfur and nitrogen permits electron stabilization of the metal nanoparticles formed.

A monolith consisting of an inorganic polymer of the prior art, described for example in the aforementioned Fr-2,852,947, could not be used as a metal catalyst support, on account of the fact that the inorganic polymer constituting said monolith of the prior art possesses only silanol groups on the surface that do not enable heterogeneous nucleation of the metal nanoparticles to be induced.

A material according to the present invention may also be useful for a Mitzoroki-Heck reaction, which is a carbon-carbon coupling reaction that makes it possible to form a biphenyl compound from an aryl halide (1) and styrene (2). Said reaction gives a mixture of E and Z isomers of stilbene. The halogen is chosen from Cl, Br and I. The equation for the reaction is given below for an iodide.

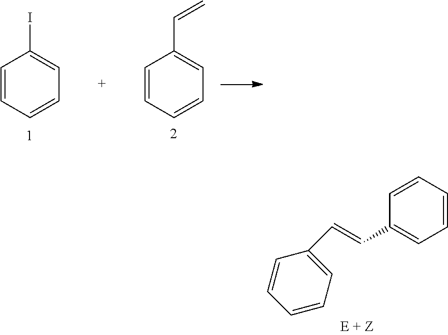

When the R substituent of a hybrid material according to the present invention is a lower alkyl group (1 to 3 carbon atoms) or a phenyl group, the hybrid material has a large capacity to adsorb aromatic compounds such as benzene, toluene or xylene (called hereinafter “BXT compounds”). It is therefore particularly useful for the decontamination of liquid or gaseous media that contain these compounds.

When the medium to be contaminated is a liquid medium, decontamination is carried out by immersion of the hybrid material in the liquid to be decontaminated.

When the medium to be decontaminated is a gaseous medium, the hybrid material is placed in a chamber, for example a column, and the gas to be decontaminated is led through the chamber.

A monolith of the prior art that possesses silanol groups on the surface, has a hydrophilic character to a high degree, which notably limits the impregnation of the the monolith by hydrophobic liquids such as benzene, xylene or toluene.

### DESCRIPTION OF THE DRAWINGS

FIG. 1a is a photograph by transmission electron microscopy (TEM) of the general appearance of an SiO₂ monolith containing 3-pyrrolylpropyl groups denoted by pyrrole-SiO-1a.

FIGS. 1b to 1g represent photographic plates obtained by TEM, for pyrrole-SiO-1a, methyl-SiO-1a, Benzyl-SiO-2a and Mercapto-SiO-1a monoliths containing respectively 3-pyrrolylpropyl groups (plate 1b) methyl groups (plate 1c), 3-(2,4-dinitrophenylamino) propyl groups (plate 1d), benzyl groups (plate 1e), and 3-mercaptopropyl groups (plates 1f and 1g).

FIGS. 2a to 2e represent differential intrusion (in ordinates, expressed in ml/g/nm) as a function of the diameter of the intermacropore windows (in abscissas, expressed in nm), respectively for pyrrole-SiO-1a, methyl-SiO-1a, DNP-amino-SiO-1a, Benzyl-SiO-2a, Mercapto-SiO-1a, and g-amino-SiO monoliths.

FIGS. 3a and 3e are transmission electron microscope (TEM) photographic plates and FIGS. 3f to 3e are SAXS diffusion profiles respectively for pyrrole-SiO-1a, methyl-SiO-1a, DNP-amino-SiO-1a, Benzyl-SiO-2a and Mercapto-SiO-1a monoliths.

FIGS. 3ka to 3kb are SAXS diffusion profiles produced on the g-amino-SiO and g-mercapto-SiO monoliths.

FIGS. 4a to 4e represent the pore size distribution determined by the DFT (Differential Functional Theory) method. The results are presented in FIGS. 4a to 4e, respectively for the Pyrrole-SiO-1a, Methyl-SiO-1a, DNP-amino-SiO-1a, Benzyl-SiO-2a, Mercapto-SiO-1a, g-amino-SiO and g-mercapto-SiO monoliths. The pore width (in A) is indicated as abscissas, and the differential surface area (in m²/g) is indicated in ordinates.

FIG. 5 represents from top to bottom, the NMR spectrum of methyl-SiO-1a, mercapto-SiO-1a, benzyl-SiO-1a, pyrrole-SiO-1a and DNP-amino-1a monoliths.

FIG. 6 represents the IR spectra that show signals corresponding respectively to the N-(3-propyl)pyrrole group (1360 cm⁻¹ and 1650 cm⁻¹, FIG. 6a), to the methyl group (2856 cm⁻¹ and 2932 cm⁻¹, FIG. 6b), to the 3-(2,4 dinitrophenylamino)propyl group (1338 cm⁻¹ and 1622 cm⁻¹, FIG. 6c) to the benzyl group (4 bands between 1450 and 1650 cm⁻¹, FIG. 6d) and to the 3-mercaptopropyl group (690 cm⁻¹, FIG. 6e).

FIGS. 7a to 7f represent the TEM plates for catalytic systems consisting of a monolith and palladium, respectively for the Pd@g-AE-amino-SiO, Pd@g-Amino-SiO, @-Mercapto-SiO, Pd@g-Mercapto-SiO, Pd@mercapto-SiO-1a, Pd@g-DNP-amino-SiO and Pd@g-pyrrole-SiO systems.

FIGS. 8a and 8b represent the XPS diagrams of monoliths carrying N-(2-aminoethyl)3-amino-propyl) groups and Pd particles generated by heterogeneous nucleation. FIG. 8b is an enlargement of the X-ray emission bands specific to palladium.

FIG. 9 represents the XPS diagram of a Pd@g-amino-SiO monolith carrying N-(2-aminoethyl) 3-amino-propyl), groups and Pd particles generated by heterogeneous nucleation.

FIGS. 10a to 10f show the degree of conversion obtained in a Suzuki-Myaura reaction with each of the following catalytic systems: Pd@g-AE-amino-SiO (10a), Pd@g-Mercapto-SiO (10b), Pd@g-Mercapto-SiO (10c), Pd@g-pyrrole-SiO (10d), Pd@g-AE-amino-SiO (10e) and Pd@g-Amino-SiO (10f).

FIG. 11 shows the degree of conversion obtained for the same Suzuki-Myaura reaction with a Pd catalyst of the prior art on active carbon.

FIG. 12 shows the change in the rate of conversion C in %, during time T (in minutes), when the catalyst is used for successive cycles in the Mitzoroki-Heck reaction, for the catalysts Pd@g-amino-SiO (curve indicated by a square □), Pd@g-mercapto-SiO (curve indicated by a black circle •), Pd@mercapto-SiO (curve indicated by a triangle Δ) and Pd@g-amino-SiO (curve indicated by a circle ◯).

FIG. 13 is a curve that represents in ordinates the percentage impregnation of a monolith as a function of time in minutes (indicated as abscissas).

The present invention is illustrated by the concrete examples described hereinafter, to which it is however not limited.

Examples A1 to A 3 concern the preparation of hybrid materials according to the invention, example A4 describes the characterization of the materials obtained, examples B1 to B2 describe the preparation of supported catalysts from materials according to the invention, example C1 and C2 describe catalytic tests, and examples D1 and D2 described decontamination treatment tests.

### Example A1

Preparation of an SiO₂ Monolith Carrying R Groups, with R=Benzyl

This example illustrates the first variant of the method.

4.05 g of tetraethoxysilane (TEOS) and 1 g of benzyltriethoxysilane were added to 16.01 g of a 35% by weight aqueous solution of tetradecyltrimethylammonium bromide (TTAB). 5.87 g of 37% HCl were then added. In order to permit hydrolysis of the compounds before the oily phase was added, the solution prepared in this way was left with stirring for 5 minutes. The oily phase, consisting of 40.06 g of dodecane, was then added dropwise, and the system was then emulsified by hand with a mortar. The emulsion prepared in this way was placed in a closed plastic container in order to allow the precursors to condense. The condensation step proceeded over a period of one week. The oily phase was then extracted by immersing the compound in a THF/acetone solvent (80/20 by volume) for 24 hours. This washing step was repeated three times, before the immersed compound was left for one hour in an acetone solution. The compound was then dried by leaving it in air in a beaker with a non-airtight lid on top, in order to prevent too violent or rapid evaporation of the washing solvent that would bring about the formation of crack zones in the monolith prepared in this way. Finally, the compound was treated for 6 hours at 180° C. (temperature rise rate of 2° C. per minute), so as to sinter it slightly and in this way to improve its mechanical strength.

### Preparation of SiO₂ Monoliths Carrying Other R Groups

Other trialkoxysilanes could also be used for the preparation of SiO₂ hybrid monoliths, by following the same operating mode as described above according to the first variant of the method according to the invention. They consisted of the following AMR compounds: methyltriethoxysilane, (3-mercaptopropyl)trimethoxysilane, (3-aminopropyl) triethoxysilane, 3-(2,4-dinitrophenylamino)propyltriethoxysilane, N-(2-aminoethyl)-3 aminopropyltrimethoxysilane and N-(3-trimethoxysilylpropyl) pyrrole.

Table 1 gives, for each preparation, the weights (in grams) of tetraethoxysilane (TEOS), of the AMR compound, of TTAB, of dodecane and of the HCl used.

TABLE 1

| | Reagent | | | | | |
|---|---|---|---|---|---|---|
| | | | | | | Monolith |
| R | TEOS | AMR | TTAB | Dodecane | HCl | obtained |
| Methyl | 4.04 | 1.03 | 16.03 | 40.02 | 5.87 | Methyl- |
| | | | | | | SiO-1a |
| N-(3- | 4.05 | 1.00 | 16.05 | 40.03 | 5.9 | Pyrrole- |
| propyl)pyrrole | | | | | | SiO-1a |
| 3- | 4.01 | 1.02 | 16.03 | 40.06 | 5.89 | Mercapto- |
| mercaptopropyl | | | | | | SiO-1a |
| (3- | 4.03 | 1.01 | 16.03 | 40.02 | 5.89 | Amino- |
| aminopropyl) | | | | | | SiO-1a |
| 3-(2,4 | 4.03 | 1.01 | 16.06 | 40.06 | 5.9 | DNP- |
| dinitrophenyl- | | | | | | amino- |
| amino)propyl | | | | | | SiO-1a |
| N-(2- | 4.04 | 1.02 | 14.01 | 40.04 | 7.86 | AE- |
| aminoethyl)-3 | | | | | | amino- |
| aminopropyl | | | | | | SiO-1a |

### Example A2

Preparation of an SiO₂ Monolith Carrying R Groups with R=3-Mercaptopropyl

This example illustrates the second variant of the method.

4.02 g of TEOS were added to 16.01 g of a 35% by weight aqueous solution of tetradecyltrimethylammonium bromide (TTAB). 5.87 g of 37% hydrochloric acid were then added. In order to permit hydrolysis of the TEOS before the oily phase was added, the solution prepared in this way was left with stirring for 3 minutes. The oily phase, consisting of 40.06 g of dodecane containing 1.02 g of (3-mercaptopropyl)trimethoxysilane) was added dropwise, and the system was then emulsified by hand with a mortar. The emulsion prepared in this way was placed in a closed plastic container in order to allow the precursors to condense. The condensation step proceeded over a period of one week. The oily phase was then extracted by immersing the compound in a THF/acetone solvent (80/20 by volume) for 24 hours. This washing step was repeated three times, before the immersed compound was left for one hour in an acetone solution. The compound was then dried by leaving it in air in a beaker with a non-airtight lid on top. The compound was then treated for 6 hours at 180° C. (temperature rise rate of 2° C. per minute), so as to sinter it slightly and in this way to improve its mechanical strength.

### Preparation of SiO₂ Monoliths Carrying Other R Groups

Other trialkoxysilanes were also used for the preparation of SiO₂ hybrid monoliths, following the same operating mode as described above according to the second variant of the method according to the invention. They consisted of the following AMR compounds: methyltriethoxysilane, benzyltriethoxysilane, (3-aminopropyl)triethoxysilane, 3-(2,4-dinitrophenylamino)propyltriethoxysilane, N-(2-aminoethyl)-3-aminopropyltrimethoxysilane and N-(3-trimethoxysilylpropyl)pyrrole.

Table 2 gives, for each preparation, the weights (in grams) of tetraethoxysilane (TEOS), of the AMR compound, of TTAB, of dodecane and of the HCl used.

TABLE 2

| | Reagent | | | | | |
|---|---|---|---|---|---|---|
| | | | | | | Monolith |
| R | TEOS | AMR | TTAB | Dodecane | HCl | obtained |
| Methyl | 4.05 | 1.03 | 16.01 | 40.03 | 5.9 | Methyl- |
| | | | | | | SiO-2a |
| Benzyl | 4.00 | 1.04 | 16.02 | 40.02 | 5.89 | Benzyl- |
| | | | | | | SiO-2a |
| N-(3- | 4.03 | 1.01 | 16.07 | 40.02 | 5.9 | Pyrrole- |
| propyl)pyrrole | | | | | | SiO-2a |
| (3- | 4.08 | 1.01 | 16.00 | 40.01 | 5.98 | Amino- |
| aminopropyl) | | | | | | SiO-2a |
| 3-(2,4 | 4.02 | 1.00 | 16.05 | 40.03 | 5.99 | DNP- |
| dinitrophenyl- | | | | | | amino- |
| amino)propyl | | | | | | SiO-2a |
| N-((2- | 4.04 | 1.12 | 14.02 | 40.04 | 7.8 | AE- |
| aminoethyl)3- | | | | | | amino- |
| aminopropyl | | | | | | SiO-2a |

### Example A3

Preparation of an SiO₂ Monolith Carrying R Groups with =3-Pyrrolylpropyl. This Example Illustrates the Third Variant of the Invention.

The SiO₂ monolith was first prepared. To this end, 6.1 g of hydrochloric acid were introduced into 16.07 g of a 35% by weight TTAB solution. 5.01 g of TEOS were then added dropwise as well as 40.02 g of decane, while emulsifying by hand by means of a mortar. The condensation step for the precursor proceeded for a period of one week and the oily phase was then extracted by immersing the monolith obtained in THF for hours, this step being repeated three times. The monolith was then carefully dried, so as to avoid too violent evaporation of THF. The monolith was then calcined at 600° C. in air for 6 hours, so as to sinter it slightly and to release the mesoporosity (induced by TTAB micelles). The material constituting the monolith thus obtained is called hereinafter “native silica”.

In a second step, 3-pyrrolylpropyl groups were grafted onto the SiO₂ monolith synthesized in the first step, by proceeding in the following way: 3.1 g. of N-(3-trimethoxysilylpropyl)pyrrole were introduced into 150.40 g of chloroform. 1.2 g of the SiO₂ monolith were then immersed in this solution. In order to increase the diffusion kinetics, the beaker containing the solution and the monolith was placed in a chamber under vacuum until the monolith fell to the bottom of the beaker. It could be ensured in this way that the monolith was completely impregnated by the reaction medium. This step lasted between 5 and 10 minutes. The beaker was then taken out of the vacuum chamber and then closed and allowed to stand for 24 hours. The compound obtained was then placed for one hour in a beaker containing acetone. The monolith was then dried in air in a beaker having a non-airtight lid on top.

### Grafting of Other Compounds on an SiO₂ Monolith

Other trialkoxysilanes were also used to prepare hybrid SiO₂ monoliths, by following the same operating mode as that described above, according to the third variant of the method according to the invention. They consisted of the following compounds: methyltriethoxysilane,
benzyl-triethoxysilane,
(3-mercaptopropyl)trimethoxysilane,
(3-aminopropyl)triethoxysilane,
3-(2,4 dinitrophenylamino)propyltriethoxysilane and
N-(2-aminoethyl)-3-aminopropyltrimethoxysilane.

Table 3 gives, for each preparation, the weights (in grams) of the SiO₂ monolith, trialkoxysilane (AMR) and chloroform used.

TABLE 3

| | Reagent | | | |
|---|---|---|---|---|
| | Native | | | Monolith |
| R | silica | AMR | Chloroform | obtained |
| 3-pyrrolylpropyl | 1.2 | 3.1 | 150.40 | g-pyrrole- |
| | | | | SiO |
| Methyl | 0.57 | 1.52 | 70.3 | g-methyl- |
| | | | | SiO |
| Benzyl | 0.74 | 1.8 | 91.5 | g-benzyl- |
| | | | | SiO |
| 3-mercaptopropyl | 1.02 | 2.52 | 127.13 | g-mercapto- |
| | | | | SiO |
| 3-aminopropyl | 1.05 | 2.56 | 128 | g-amino-SiO |
| 3-(2,4 dinitrophenyl- | 1.2 | 3 | 150 | g-DNP- |
| amino)propyl | | | | amino-SiO |
| N-(2-aminoethyl)-3 | 1.35 | 3.3 | 168 | g-Ae-amino- |
| aminopropyl | | | | SiO |

### Example A4

### Characterization of the Monoliths Obtained

The monoliths obtained according to examples A1, A2 and A3 (namely according to the three variants of the method according to the invention) were characterized by various analytical methods so as to reveal their macroporous, mesoporous and microporous character. The monoliths obtained according to the first variant of the method exhibited the same properties as those obtained according to the second variant. Consequently, the data presented below for monoliths synthesized according to example A1 were acceptable for monoliths synthesized according to example A2 carrying the same R groups.

The monoliths subjected to characterization were as follows: pyrrole-SiO-1a, methyl-SiO-1a, DNP-amino-SiO-1a, mercapto-1a, benzyl-SiO-2a, mercapto-SiO-1a, g-amino-SiO and g-mercapto-SiO.

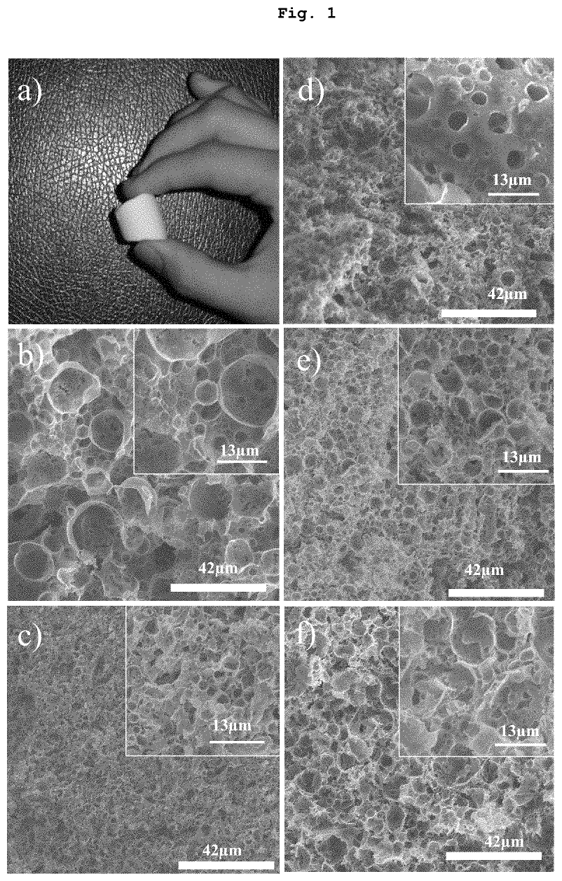

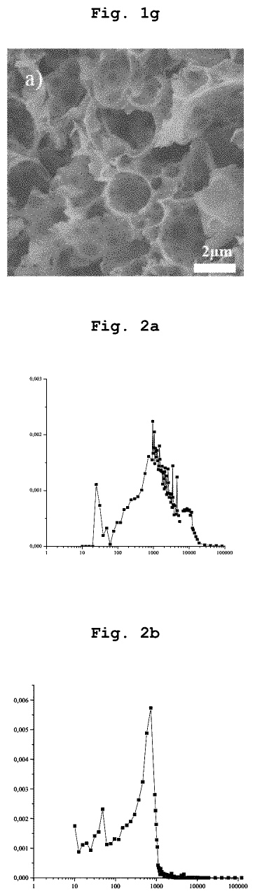

The general appearance of a monolith according to the invention is presented in the photograph of FIG. 1a. It consists of an SiO₂ monolith containing the N-(3-propyl)pyrrole groups of example A1, denoted by pyrrole-SiO-1a.

### Macroporous Structure

The photographic plates of figures 1b to 1f were obtained by transmission electron microscopy (TEM). These plates were produced on pyrrole-SiO-1a, methyl-SiO-1a, DNP-amino-SiO-1a, benzyl-SiO-2a, mercapto-SiO-1a monoliths, containing respectively 3-pyrrolylpropyl groups (plate 1b), methyl groups (plate 1c), 3-(2,4-dinitrophenylamino)propyl groups (plate 1d), benzyl groups (plate 1e) and 3-mercaptopropyl groups (plate 1f).

The plate of FIG. 1g was obtained by scanning electron microscopy (SEM) from the monolith or g-mercapto-SiO.

These plates show that the macroscopic cells were polydispersed with a size varying between 5 μm and 30 μm. The macroscopic structure of the monoliths resembled an aggregation of hollow spheres (similar to that of the native silica monolith of example A3), with the exception of the monolith having (dinitrophenylamino)propyl groups (plate 1d), of which the intercellular walls were completely mineralized.

Mercury intrusion macroporosimetry measurements were performed at ambient temperature for various samples.

The sample was weighed and degassed under a vacuum of 6×10⁻⁶ MPa, before being placed in a measuring cell. The measuring cell was then filled with mercury at a pressure of 3.4×10⁻³ MPa and then successive pressures were generated between 3.4×10⁻³ MPa and 120 MPa (which corresponded to the theoretical pore diameters). At each pressure, the electrical capacity was measured by the rod of a penetrometer and a deduction was made of the volume of mercury that had penetrated into the sample. The results are given in the table 4 below.

TABLE 4

| | | | | Density of | |
|---|---|---|---|---|---|
| | Intrusion | | | the | |
| | volume | Porosity | Density | skeleton | |
| | (cm³ · g⁻¹) | (%) | (g · cm⁻³) | (g · cm⁻³) | Curve |
| Pyrrole-SiO- | 13.7 | 96.2 | 0.07 | 1.86 | a |
| 1a | | | | | |
| Methyl-SiO-1a | 3.6 | 80 | 0.22 | 1.13 | b |
| DNP-amino- | 3.38 | 92 | 0.27 | 1.64 | c |
| SiO-1a | | | | | |
| Benzyl-SiO-2a | 5.12 | 89.4 | 0.17 | 1.65 | d |
| Mercapto-SiO- | 9.92 | 93 | 0.09 | 1.36 | e |
| 1a | | | | | |
| g-amino-SiO | 3.74 | 88 | 0.24 | 1.93 | |
| g-mercapto- | 10.65 | 92 | 0.09 | 1.07 | |
| SiO | | | | | |

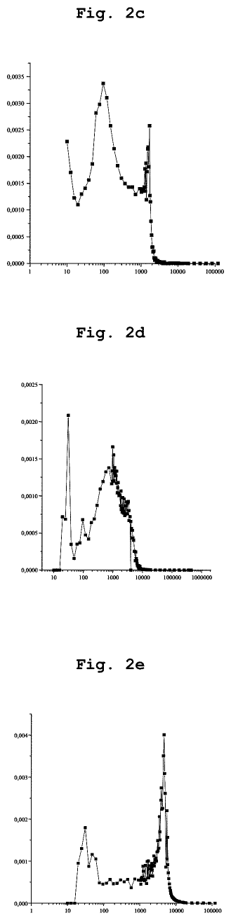

The results of mercury intrusion porosimetry measurements have been given in FIG. 2 which represents, for each sample, the differential intrusion (in ordinates, expressed in ml/g/nm) as a function of diameter of the intermacropore windows (in abscissas, expressed in nm). As the case may be, the curve corresponding to each monolith is referred to in the last column of the above table.

It follows from these measurements that the windows connecting two adjacent macropores have a bimodal character. These windows and the associated macropores correspond to the characteristic sizes that permit impregnation with and rapid flow of solvent within the material (Darcy's law). These interconnected macropores (by inter-pore windows) will make it possible to irrigate all the mesopores and in this way to optimize all the surface area of the materials, which constitutes an important property for impregnation by BXT compounds.

### Mesoporous and Microporous Character

The mesoporous character was studied by transmission electron microscopy associated with small angle X-ray diffraction measurements (SAXS).

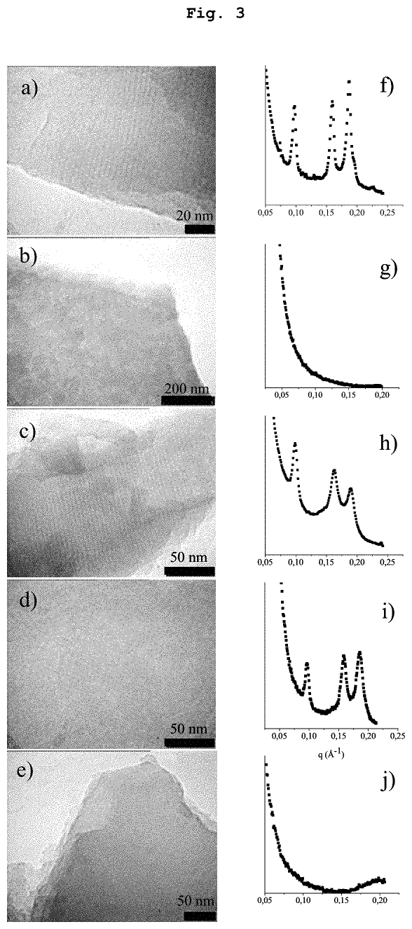

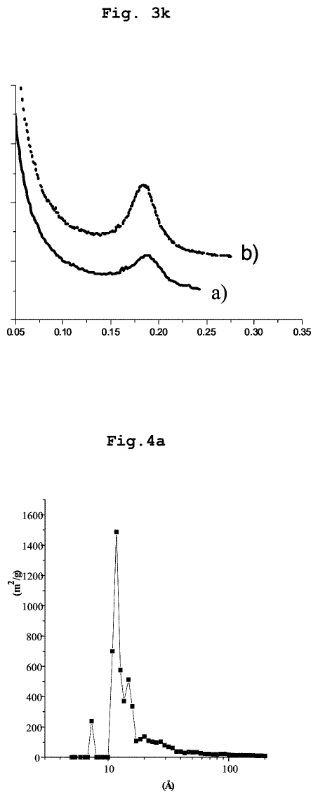

FIGS. 3a to 3e are transmission electron microscopy plates (TEM) produced on SiO₂ monoliths. FIGS. 3f to 3e are SAXS diffusion profiles performed on the same samples. Intensity is given as ordinates (arbitrary units), as a function of the wave vector q (in Å⁻¹).

FIGS. 3ka to 3 kb are SAXS diffusion profiles performed on other samples.

The correspondence between the monoliths and curves of FIG. 3 given in the following table.

| Monolith | MET plate | SAXS curve |
|---|---|---|
| Pyrrole-SiO-1a | a | f |
| Methyl-SiO-1a | b | g |
| DNP-amino-SiO-1a | c | h |
| Benzyl-SiO-2a | d | i |
| Mercapto-SiO-1a | e | j |
| g-amino-SiO | | ka |
| g-mercapto-SiO | | kb |

It follows that all the materials exhibited a mesoporous character. The figures also show that
mesopores were dispersed statistically for the methyl-SiO-1a monoliths (plate 3b and FIG. 3g) and the mercapto-SiO-1a monoliths (plate 3e and FIG. 3j);
the mesopores were organized in a hexagonal manner in the pyrrole-SiO-1a, DNP-amino-SiO-1a and benzyl-SiO-2a monoliths;
in the g-amino-SiO and g-mercapto-SiO monoliths, the mesopores had a polydisperse distribution extending from 10 to 6000 nm, with two main contributions centred on 150 nm and 700 nm for g-amino-SiO, and centred on 60 nm and 4000 nm for g-mercapto-SiO.

Specific surface area measurements were also performed by nitrogen adsorption-desorption techniques (B.E.T. and B.J.H. methods). The results are given in table 5 below.

TABLE 5

| | Specific | Specific | |
|---|---|---|---|
| | surface area | surface area | |
| | (m² · g⁻¹) by | (m² · g⁻¹) by | Total pore |
| | B.E.T | B.J.H | volume |
| Pyrrole-SiO-1a | 392 | 83 | |
| Methyl-SiO-1a | 450 | 98 | |
| DNP-amino-SiO- | 217 | 87 | |
| 1a | | | |
| Benzyl-SiO-2a | 107 | 14 | |
| Mercapto-SiO-1a | 53 | 15 | |
| Native SiO₂ | 725 | 220 | 0.34 |
| g-amino-SiO | 73 | 28 | 0.07 |
| g-mercapto-SiO | 381 | 63 | 0.19 |

From the results of table 5, it may be concluded that the monoliths had a super-microporous character (pore size between 10 and 20 Å) as well as a mesoporous character (pore size greater than 35 Å). These results confirm that the grafting of organic groups onto the surface of the pores reduces the specific surface area and the pore volume compared with native silica.

The BJH method essentially gave mesopores having a size greater than 35 Å. The microporosity was obtained by difference with the BET data. The pore size distribution, obtained by the theory of differential functions, gave a bimodality of the pore sizes centered on 15 Å (super-micropores) and 25 Å (mesopores).

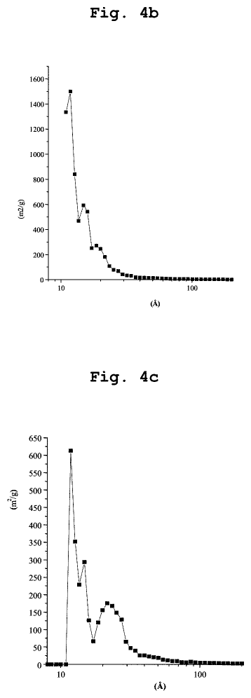

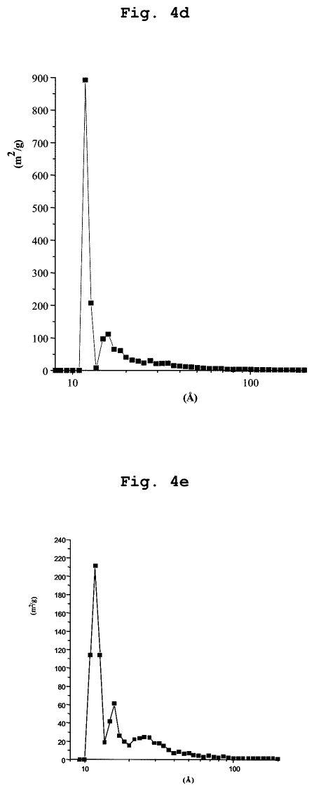

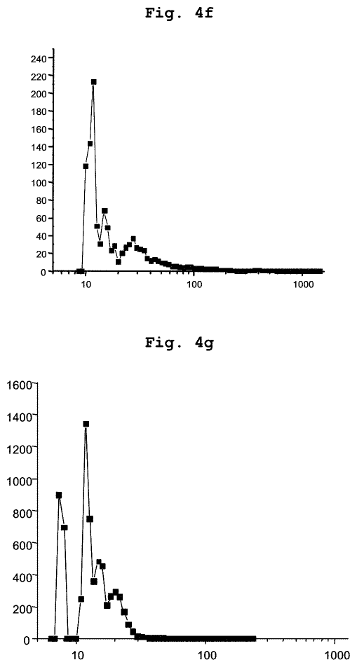

The pore size distribution was also determined by the DFT (differential functional theory) method. The results are given in FIGS. 4a to 4g. These figures represent the pore width (in A) in abscissas, and the differential surface area (in m²/g) in ordinates for the above monoliths.

The correspondence between the monoliths and the figures is given in the table below.

| Monolith | FIG. 4 |
|---|---|
| Pyrrole-SiO-1a | a |
| Methyl-SiO-1a | b |
| DNP-amino-SiO-1a | c |
| Benzyl-SiO-2a | d |
| Mercapto-SiO-1a | e |
| g-amino-SiO | f |
| g-mercapto-SiO | g |

The results that follow from these figures are in good agreement with the results obtained by the BET and BJH methods since, for all the samples, the curves exhibited a bimodal character with a peak around 10 Å (presence of micropores) and a peak around 22 Å (presence of mesopores).

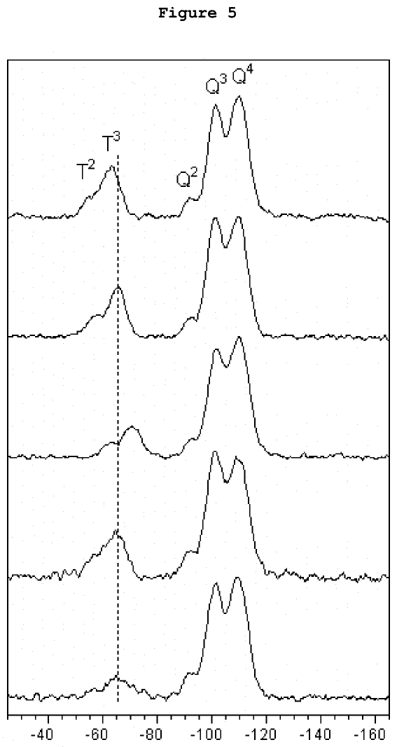

The microporous character of the monoliths was also studied by NMR ²⁹Si measurements, of which the results are given in FIG. 5.

The spectra correspond, from top to bottom, to the methyl-SiO-1a, mercapto-SiO-1a, benzyl-SiO-1a, pyrrole-SiO-1a, and DNP-amino-1a monoliths.

The T and Q peaks of the spectra are attributed as indicated in table 6.

TABLE 6

| | | | T³: −63/− | T2: −55/− |
|---|---|---|---|---|
| Q⁴: −109 ppm | Q³: −100 ppm | Q²: −92 ppm | 70 ppm | 62 ppm |
| Si(OSi)₄ | HO-Si(OSi)₃ | (HO)₂- | R-Si (OSi)3 | (HO)R- |
| | | Si(OSi)₃ | | Si(OSi)₂ |

This method made it possible to identify and quantify the various siloxane groups present in the monoliths. Table 7 gives a comparison of the results obtained from NMR ²⁹Si measurements with the expected results from the molar ratios of the precursors of the reaction (TEOS and alkoxysilane groups).

TABLE 7

| | | % | | |
|---|---|---|---|---|
| R ↓ | % TEOS | trialkoxysilane | % Q units | % T units |
| Methyl-SiO- | 77.0 | 23.0 | 78.0 | 22.0 |
| 1a | | | | |
| Mercapto- | 78.8 | 21.2 | 79.0 | 21.0 |
| SiO-1a | | | | |
| Benzyl-SiO- | 82.5 | 17.5 | 83.3 | 16.7 |
| 1a | | | | |
| Pyrrole- | 81.4 | 18.6 | 79.5 | 20.5 |
| SiO-1a | | | | |
| DNP-amino- | 88.1 | 11.9 | 89.1 | 10.9 |
| 1a | | | | |

The experimental results (two right hand columns) were in agreement with the theoretical calculations (two left hand columns) which show that the synthetic method used made it possible to control well the final composition of the material.

Moreover, infrared spectroscopy measurements were taken so as to verify that the final treatment of the monoliths at 180° C. for 6 hours had not damaged the R groups.

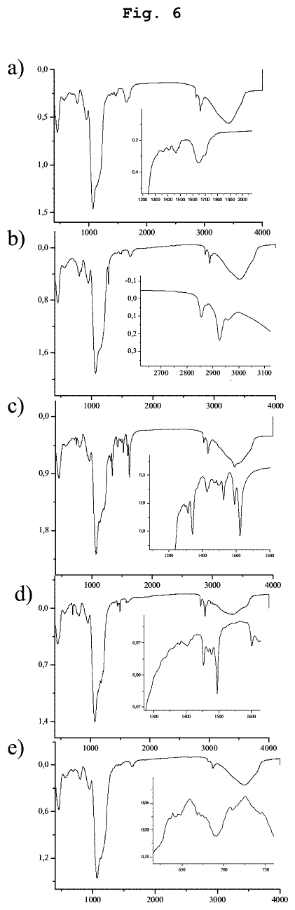

The spectra obtained are shown in FIGS. 6a to 6e. The correspondence between the monoliths and the figures is given in the table below.

| Monolith | FIG. 6 |
|---|---|
| Pyrrole-SiO-1a | a |
| Methyl-SiO-1a | b |
| DNP-amino-SiO-1a | c |
| Benzyl-SiO-2a | d |
| Mercapto-SiO-1a | e |

These spectra show the signals corresponding respectively to the 3-pyrrolylpropyl group (1360 cm⁻¹ and 1650 cm⁻¹, FIG. 6a), the methyl group (2856 cm⁻¹ and 2932 cm⁻¹, FIG. 6b), the 3-(2,4-dinitrophenylamino)propyl group (1338 cm⁻¹ and 1622 cm⁻¹, FIG. 6c), the benzyl group (4 bands between 1450 cm⁻¹ and 1650 cm⁻¹, FIG. 6d) and the 3-mercaptopropyl group (690 cm⁻¹, FIG. 6e).

The R groups present in the monoliths were thus not damaged by the effect of heat treatment.

### Example B1

The supported catalysts were prepared from materials obtained according to the method of example A3, and carrying respectively N-(2-aminoethyl)-3-aminopropyl, 3-aminopropyl, 3-mercaptopropyl, 3-(2,4-dinitrophenylamino)propyl and N-(3-propyl)pyrrole groups and a material carrying 3-mercaptopropyl groups prepared according to the method of example 1.

### Synthesis

A hybrid monolith obtained according to the method of example A3 was impregnated with a 5×10⁻² M solution of Pd(CH₃COO)₂ in THF for a period of two days, while employing three degassing cycles of 15 minutes each, and a 0.5 M NaBH₄ solution was then added in a water/THF mixture (50/50). This mixture was allowed to stand for one day using the same degassing cycles as previously, and the materials were then recovered by filtration, washed with an ethanol/acetone mixture (80/20 by volume) for 24 hours with stirring, and dried in the open air.

The following table indicates the catalytic systems prepared and the modified monolith from which each one was derived.

| Name of the catalytic system | Initial monolith |
|---|---|
| PD@g-AE-amino-SiO | g-AE-amino-SiO (example 3) |
| PD@g-Amino-SiO | g-Amino-SiO (example 3) |
| PD@g-Mercapto-SiO | g-Mercapto-SiO (example 3) |
| PD@Mercapto-SiO-1a | Mercapto-SiO (example 1) |
| PD@g-DNP-amino-SiO | g-DNP-amino-SiO (example 3) |
| Pd@pyrrole-SiO | g-pyrrole-SiO (example 3) |

### Characterization by TEM

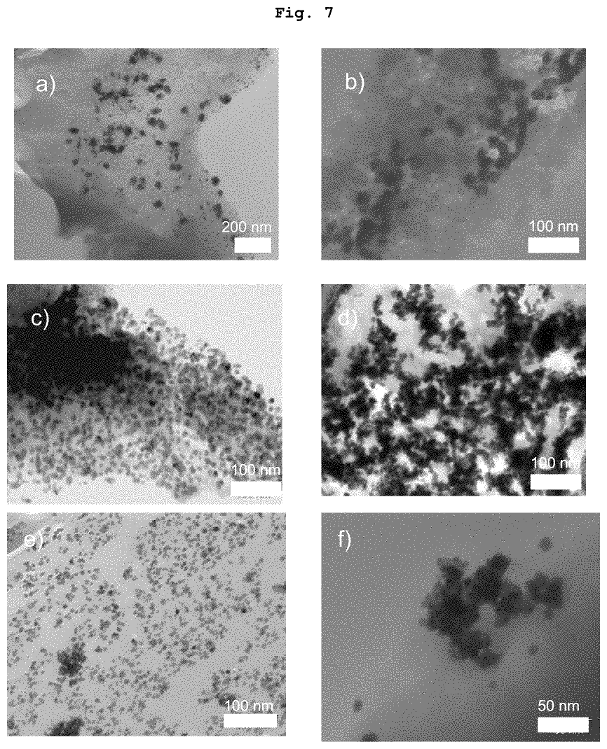

The catalytic systems obtained were characterized by transmission electron microscopy. FIG. 7 shows the TEM photographic plates obtained. The correspondence between various plates and the catalytic systems is given in the following table.

| Catalytic system | Plate |
|---|---|
| PD@g-AE-amino-SiO | 7a |
| PD@g-Amino-SiO | 7b |
| PD@g-Mercapto-SiO | 7c |
| PD@Mercapto-SiO-1a | 7d |
| PD@g-DNP-amino-SiO | 7e |
| Pd@pyrrole-1a | 7f |

The monoliths used were obtained by the method of example 3, except for the PD@Mercapto-SiO₂ monolith of FIG. 7d which was obtained by the method of example 1.

These plates gave important information on the degree of aggregation of the supported catalysts, knowing that an increase in the degree of aggregation corresponds to a reduction in the active surface area and consequently the catalytic efficiency.

### Example B2

Supported catalysts were prepared from the same hybrid monoliths as those indicated in example B1, in the presence of triphenylphospine.

### Synthesis

Pd(CH₃COO)₂ (0.33 g, 1.5 mmol) was dissolved in 30 ml of THF in order to obtain a concentration of 5×10⁻² mol·1⁻¹. Triphenylphosphine was then added (two equivalents, 3 mmol, 0.78 g). The mixture was stirred until completely dissolved. A change of color was then observed, the solution passing from a brown color to a bright red color. A 0.8 g quantity of hybrid material was added and three degassing cycles of 15 minutes each were carried out for three days so as completely to impregnate the hybrid material.

A freshly prepared solution of NaBH₄ (10 equivalents, 0.56 g, 15 mmol) in 30 ml of a water/THF mixture (50/50 v/v), was added to the solution containing the hybrid material with gentle stirring. The solution became black.

The blocks of hybrid material were recovered by filtration, washed for two days with ethanol with stirring and then dried in the open air.

### Characterization by TEM

The TEM plates obtained were similar to those for materials prepared according to example B1.

### Characterization by XPS

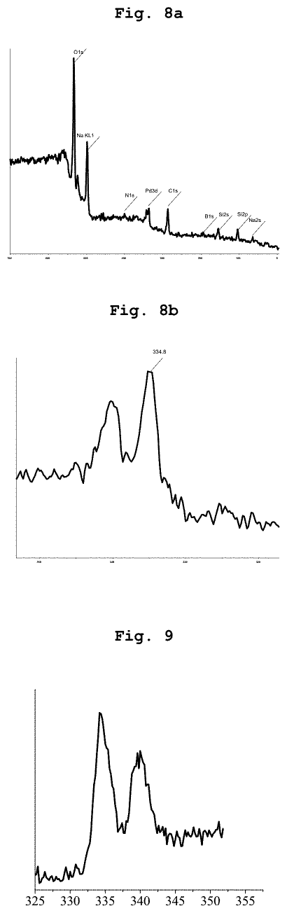

FIGS. 8a and 8b represent the XPS diagrams of monoliths carrying N-(2-aminoethyl)3-amino-propyl groups and Pd particles generated by heterogeneous nucleation. FIG. 8a, which shows an extended energy range, shows the elements present within the aforementioned compound. FIG. 8b is an enlargement of the X-ray emission bands particular to palladium, and in particular peaks of electrons associated with the 3d_{5/2} and 3d_{7/2} orbitals. The 3d_{5/2} band was centered on 335 eV and the 3d_{7/2} band was centered on 340 eV. Such energies associated with the 3d_{5/2} and 3d_{7/2} electrons were characteristic of metallic palladium in the zero valence state (non oxidized) from the publication by Brun, M., Berthet, A., Bertolini, J. C.: XPS, ARS and Auger parameter of Pd and PdO, J. Electron Microsc. Relat. Phenom., 1999, vol. 104, p 55.

The palladium content was determined by elementary analysis for the sample of material carrying mercapto groups. It was 3.9% by weight.

### Example B3

Supported catalysts were prepared from the hybrid monoliths prepared according to example 3.

### Synthesis

1 g of the monolith obtained according to example A3 was added to a solution containing Pd(CH₃COO)₂ (0.33 g, 1.5 mmol) and triphenylphosphine PPh₃ (4 equivalents, 6 mmol, 1.57 g) in 30 ml of THF to obtain a concentration of 5×10⁻² mol·l⁻¹ of acetate, and was left in the dark for 2 days.

A freshly prepared solution of NaBH₄ (10 equivalents, 0.56 g, 15 mmol) in 30 ml of a water/THF mixture 50/50 v/v), was added to the solution containing the hybrid material with gentle stirring. The color of the reaction medium changed from yellow to black in one hour.

The monolith of hybrid material was then recovered by filtration, washed for two days with ethanol until it became colorless and then dried in the open air.

A supported catalyst was prepared in this way, on the one hand with a g-amino-SiO monolith and on the other with a g-mercapto-SiO monolith.

Characterization with TEM

The TEM plates obtained were similar to those of materials prepared according to example B1.

### Characterization by XPS

FIG. 9 represents the XPS diagram of the Pd@g-amino-SiO monolith carrying N-(2-aminoethyl)3-aminopropyl groups and Pd particles generated by heterogeneous nucleation. This diagram shows two peaks, at 335 eV and 340.8 eV, which correspond to electrons associated with the 3d_{5/2} and 3d_{7/2} orbitals of metallic palladium particles.

A Pd@g-mercapto-SiO monolith carrying mercaptopropyl groups and Pd particles generated by heterogeneous nucleation were obtained according to the same method, and its XPS diagram was similar to that of the Pd@g-amino-SiO monolith.

### Elementary Analysis

The Pd content of the supported catalyst was determined by elementary analysis. It was 3.9% by weight for the sample carrying Pd@g-amino-SiO groups and 4.1% by weight for the sample carrying Pd@g-mercapto-SiO groups.

### Example C1

The catalytic activity of the various catalytic systems obtained according to examples B1 and B2 was tested on the Suzuki-Myaura reaction, employing the following operating procedure.

A 50 ml three-necked flask was used provided with a condenser at −20° C.

1 equivalent (0.097 g) of the catalytic system was introduced into the flask as well as 200 equivalents (0.576 g) of K₂CO₃, an internal standard and 5 mL of dioxane.

A mixture was prepared of 100 equivalents (0.3905 g) of iodobenzene, 150 equivalents (0.3584 g) of phenylboronic acid and 5 mL of dioxane, and this mixture was introduced into the three-necked flask with the aid of a syringe. The three-necked flask was then left in an oil bath at 115° C. under reflux with dioxane for 3 days and a follow-up was carried out by taking samples at regular intervals.

Assessments of the state of the reaction were established by liquid phase chromatography, bringing the temperature from 50° to 180° C. at a rate of 6° C. a minute on a Varian 3300, using an injector at 220° C., a detector at 200° C. at a pressure of 10 psi, a DB5 column that had a length of 30 m, an internal diameter of 0.25 mm and a film that had a thickness of 0.1 μm.

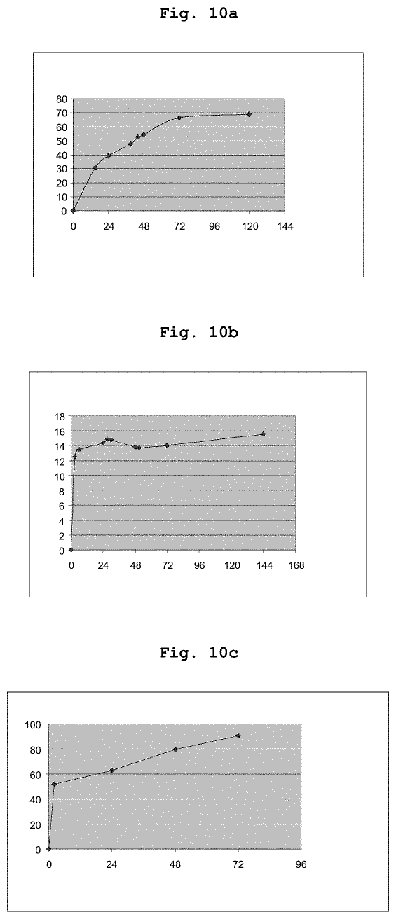

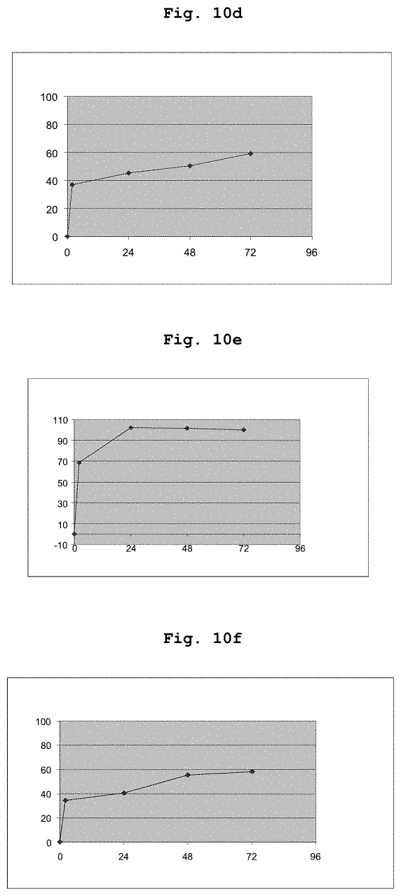

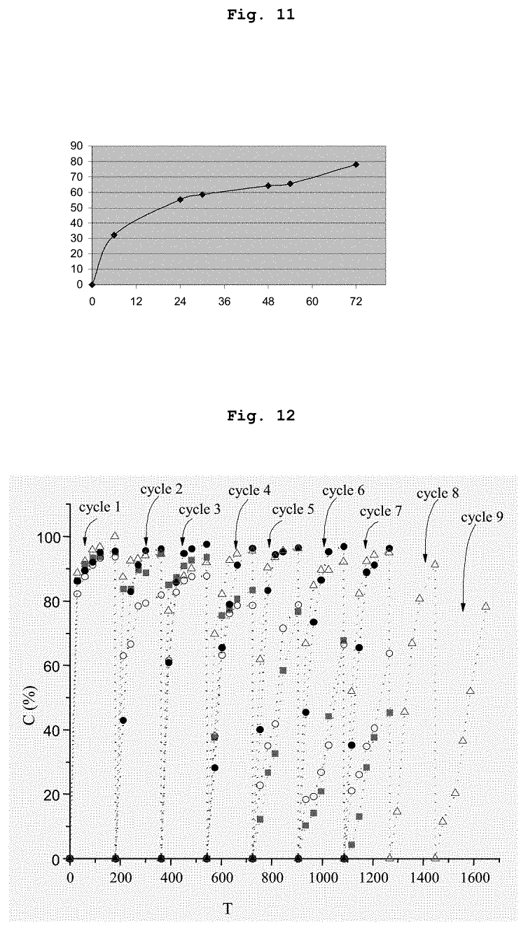

The degree of conversion obtained with each of the catalytic systems is shown as a function of time on FIGS. 10 and 12. As a comparison, the degree of conversion as a function of time for a conventional Pd catalyst on active carbon (considered as very efficient) is given in FIG. 11. On each of the figures, the degree of conversion, in percentage, is indicated as ordinates and the time (in hours) is indicated as abscissas.

The catalysts according to the invention that were tested are indicated in the following table with the corresponding figures:

| | Prepared | |
|---|---|---|
| Catalytic system | according to | Figure |
| Pd@g-AE-amino-SiO | Example B1 | 10a |
| Pd@g-Mercapto-SiO | Example B1 | 10b |
| Pd@g-Mercapto-SiO | Example B2 | 10c |
| Pd@g-pyrrole-SiO | Example B2 | 10d |
| Pd@g-AE-amino-SiO | Example B2 | 10e |
| Pd@g-Amino-SiO | Example B2 | !0f |

It appears that the catalytic systems according to the invention obtained by the method of, example B1 (without phosphine) possessed an activity close to that obtained by palladium nanoparticles on active carbon. They had however the advantage of being in a monolithic form and therefore not requiring a separation step with the catalyzed material by filtration or centrifugation for example. The materials tested thus possessed satisfactory performance and were more easily employed than a conventional catalyst such as the palladium/active carbon system.

It moreover appears that the material according to the invention obtained by the method of B2, that is to say in the presence of triphenylphosphine, possessed superior activity to that obtained by palladium nanoparticles on active carbon.

### Example C2

The catalytic activity of the various materials obtained according to examples B2 and B3 were tested on the Mitzoroki-Heck reaction, that may be shown schematically in the following manner

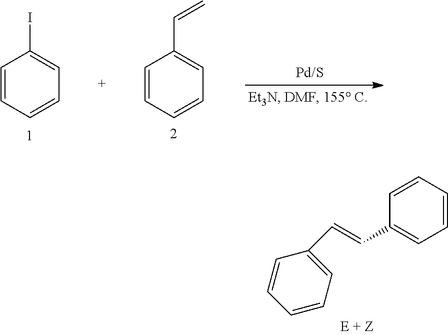

E and Z denoting isomers of stilbene.

A solution containing 10 mmol, (2.04 g) of iodobenzene 1, 15 mmol (1.56 g) of styrene 2, 11 mmol (1.11 g) of triethylamine, 5 mmol (0.85 g) of dodecane (as a reference standard for gas chromatography) and 10 mL of DMF and the supported catalyst were placed in a glass flask provided with a tap with sintered glass. The reaction medium was purged with argon for 10 min, and then the reactor was placed in an oil bath at 155° C., without stirring. Samples were extracted periodically and diluted with THF at 0° C., so as to follow the degree of conversion.

After the reaction had finished, the liquid phase was extracted from the reactor, under argon, through the sinter, a new mixture of reagents was introduced into the reactor and a new reaction was carried out. This operation was reproduced several times in order to test the stability of the catalyst with time.

The operations above were carried out on the one hand with a monolith bearing mercaptopropyl groups and on the other hand with a monolith carrying aminopropyl groups.

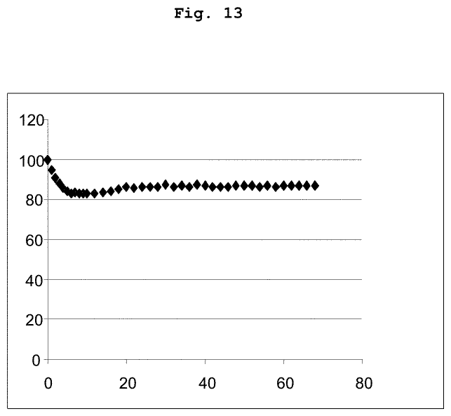

FIG. 13 shows the change in the degree of conversion with time, while the catalyst is used for successive reaction cycles, for the following catalysts:
Pd@g-amino-SiO (0.11 g): curve indicated by a square □;
Pd@g-mercapto-SiO (0.11 g): curve indicated by black circle •;
Pd@mercapto-SiO (0.11 g): curve indicated by a triangle Δ;
Pd@-amino-SiO (0.055 g): curved indicated by a circle ◯.

For the preparation of this catalyst, 0.055 g of support were used instead of 0.11 g. This was the same catalyst, but half the amount was used.

FIG. 13 shows that the catalysts gave a similar degree of conversion during their first use, which proceeded over 3 hours, and that the catalyst carrying mercapto groups and obtained from a monolith prepared according to either of samples A1 or A2 had a more stable activity during subsequent use than catalysts carrying amino groups or the catalyst carrying mercapto groups obtained by impregnation.

### Example D1

An SiO₂ monolith containing methyl groups, obtained by the method described in example A3 was used for the decontamination of a gas flow containing toluene.

0.1021 g of said monolith was used for treating a gas flow containing 241.8 mg of toluene in 1 g hexane. These proportions corresponded to a toluene level close to that generally encountered in the atmosphere, namely 10 μg/m³. Hexane was used as a carrier for toluene by reason of its quite high saturated vapor pressure, preventing it from condensing on the walls and on account of the fact that it is transparent in UV-visible.

The percentage impregnation of the monolith by toluene was estimated by UV-visible spectroscopy. The absorption band for toluene in the UV-visible is situated at 268.2 nm.

FIG. 14 is a curve that represents in ordinates the percentage impregnation of the monolith by toluene, as a function of time, in minutes, (indicated as abscissas).

It appeared that 80% of toluene contained in the gas flow was absorbed by the monolith and that this phenomenon was stable with time, since it proceeded in the same way for more than an hour.

It should be noted that this result, although convincing, is not optimal on account of the fact that the measuring chamber was not totally filled with the monolith, but contained pieces of monolith separated by empty spaces.

Similar results were obtained with hybrid monoliths containing methyl or phenyl groups, synthesized according to the methods of examples A1 and A2.

### Example D2

An SiO₂ monolith containing phenyl groups, obtained by the method described in the example A3, was used for the decontamination of a liquid phase consisting of toluene.

The hybrid monolith became opalescent after one hour's immersion in the liquid phase containing toluene. The monolith was therefore not dissolved, but took the refractive index of the surrounding medium, which showed that it had been impregnated by toluene. This phenomenon came from the special porous character of the monolith (triple porosity), of its hydrophobic character induced by phenyl groups, and the inorganic Si—O—Si connectivity that insured cohesion of the porous edifice.

Similar results were obtained with hybrid monoliths containing methyl or phenyl groups, synthesized according to the methods of examples A1 and A2.

1. A material in the form of a solid cellular monolith comprising a polymer of an inorganic oxide, wherein:
said cellular monolith has macropores having a mean size d_{A} from 4 μm to 50 μm, mesopores having a mean size d_{E} from 20 to 30 Å and micropores having a mean size d_{I} from 5 to 10 Å, said pores being interconnected;
the inorganic oxide polymer carries organic R groups corresponding to the formula —(CH₂)ₙ—R¹ in which 0≤n≤5, and R¹ represents a thiol group, a pyrrolyl group, an alkyl group, an amino group that may carry one or more possibly substituted alkyl, alkylamino or aryl substituents, or a phenyl group that may carry an alkyl substituent.

2. The material as claimed in claim 1, wherein the inorganic oxide is an oxide of one or more elements, at least one of these elements being of the type capable of forming an alkoxide.

3. The material as claimed in claim 2, wherein at least one of the metals is chosen from Si, Ti, Zr, Th, Nb, Ta, V, W and Al.

4. The material as claimed in claim 2, wherein the oxide is a mixed oxide additionally containing B and Sn.

5. The material as claimed in claim 1, wherein the inorganic polymer is a polymer of silicon oxide or a mixed oxide of silicon.

6. The material as claimed in claim 1, wherein R¹ is an alkyl group having 1 to 5 carbon atoms.

7. The material as claimed in claim 1, wherein the inorganic oxide polymer carries a single type of R group.

8. The material as claimed in claim 1, wherein the inorganic oxide polymer carries at least two different types of R group.

9. The material as claimed in claim 1, wherein the organic group R is a 3-mercaptopropyl group, a 3-aminopropyl group, a 3-pyrrolylpropyl group, an N-(2-aminoethyl)-3-aminopropyl group, a 3-(2,4 dinitrophenylamino)propyl group, a phenyl group, a benzyl group or a methyl group.

10. A method for preparing a material as claimed in claim 1, wherein an emulsion is prepared by adding an oily phase to an aqueous solution of surfactant, at least one tetra-alkoxide (TAM) precursor of the inorganic oxide polymer is added to the aqueous surfactant solution, before or after preparing the emulsion, the reaction mixture is allowed to stand until the precursor condenses, and then the mixture is dried so as to obtain a monolith, wherein within said method at least one precursor alkoxide carrying an organic R group (compound AMR) is added.

11. The method as claimed in claim 10, wherein the alkoxide AMR is introduced into the aqueous surfactant solution before the oily phase is added.

12. The method as claimed in claim 10, wherein the alkoxide AMR is introduced into the oil phase that is then added to the aqueous TAM solution to form the emulsion.

13. The method as claimed in claim 10, wherein the inorganic monolith obtained from the aqueous surfactant solution and TAM after drying is impregnated with a solution of AMR.

14. The method as claimed in claim 11, wherein the hybrid monolith obtained at the end of the drying step is subjected to a heat treatment.

15. The method as claimed in claim 10, wherein the mass ratio (alkoxide AMR/tetra-alkoxide TAM) is less than 20/80.

16. The method as claimed in claim 10, wherein the tetra-alkoxide TAM is a silicon tetraethoxysilane.

17. The method as claimed in claim 16, wherein the tetra-alkoxide TAM is tetramethoxysilane or tetraethoxysilane.

18. The method as claimed in claim 10, the alkoxide AMR is a trialkoxysilane chosen from:
3-mercaptopropyl)trimethoxysilane,
3-aminopropyl)triethoxysilane,
N-(3-trimethoxysilylpropyl)pyrrole,
3-(2,4 dinitrophenylamino)propyltriethoxysilane,
N-(2-aminoethyl)-3-aminopropyltrimethoxysilane, phenyltriethoxysilane; and
methyltriethoxysilane.

19. The method as claimed in claim 10, wherein the oily phase is chosen from dodecane or a silicone oil.

20. The method as claimed in claim 10, wherein the surfactant compound is a cationic surfactant and the reaction medium is brought to a pH below 3.

21. The method as claimed in claim 10, wherein the surfactant compound is an anionic surfactant and the reaction medium is brought to a pH above 10.

22. The method as claimed in claim 10, wherein the surfactant compound is a non-ionic surfactant and the reaction medium is brought to a pH above 10 or below 3.

23. A use for a material as claimed in claim 1 for the elimination of benzene, toluene or xylene contained in a liquid or gaseous medium.

24. A catalytic system comprising a support and a metal catalyst, wherein the support is a material as claimed in claim 1.

25. The catalytic system as claimed in claim 24, wherein the metal catalyst is in the form of nanoparticles.

26. A use for a catalytic system as claimed in claim 24, for the catalysis of a carbon-carbon coupling reaction to form a biphenyl compound according to the Mitzoroki-Heck reaction or according to the Suzuki-Myaura reaction.
